(19) United States
(12) Patent Application Publication
(10) Pub. No.: US 2013/0060201 A1
(43) Pub. Date: Mar. 7, 2013

(54) PUNCTURING DEVICE WITH A NEEDLE SHIELD AND A METHOD FOR NEEDLE SHIELD REMOVAL

(71) Applicant: Sergey Popov, Beer-Sheva (IL)

(72) Inventor: Sergey Popov, Beer-Sheva (IL)

(21) Appl. No.: 13/582,458

(22) PCT Filed: Feb. 27, 2011
(86) PCT No.: PCT/IL11/00192

§ 371 Date: Nov. 13, 2012

Related U.S. Application Data
(60) Provisional application No. 61/309,010, filed on Mar. 1, 2010.

(51) Int. Cl. A61M 5/32; A61M 5/00

(52) U.S. Cl. 604/198; 604/263

(57) ABSTRACT

Puncturing device FIG. 6 with needle shield 2, having a needle subassembly 1 and a needle shield 2 detachably mounted on the needle subassembly 1 for enclosing the needle distal end 9 up to the device usage, wherein said needle shield 2 has an actuating means 17 which being moved to a retracted position disengages the needle shield 2 from the needle assembly 1, wherein the detaching movement of the actuating means 17 is significantly shorter than the length of the needle 8.

### FIELD OF THE INVENTION

The present invention relates to medical devices and, in particular, to medical devices which include a sharp needle for piercing the skin of a patient.

### BACKGROUND OF THE INVENTION

A hypodermic needle before use stays enclosed by a shield which prevents the damage of the needle sharp tip and the damage of the sterile package. All known shields are removed from the needle by pulling them in the distal direction. This way of shield removal has some disadvantages, such as the possibility to remove the shield only by two-hand technique and a high risk to damage the device itself.

Every device equipped with a sharp needle has a handle, but some kinds of them include an additional intermediate detail placed between the shield and the handle. This intermediate detail may be, for example, a catheter hub in IV catheter placement devices or a protector in safety hypodermic needles. Very often the shield in these devices is placed on the intermediate detail, which is the catheter hub or the protector in safety hypodermic needles. And if, in such devices, the shield is removed, as usual, by pulling it in distal direction, the intermediate detail (needle protector in U.S. Pat. No. 4,790,828) may be pulled with the shield, which will results in the unfitness of the device. To preclude such result, the user has to pull the shield by one hand and to hold the intermediate detail by the other, but this is not simple in many cases. To preclude the risk of damaging the device during shield removal, sophisticated needle shields were developed (U.S. Pat. No. 5,000,740), which are mounted on the device handle. But such shields have complicated geometry and still can't be used, for example, with IV catheters having an injection port. Furthermore, the shields that are developed to be mounted on the handle always have a unique design.

### SUMMARY OF THE INVENTION

In one embodiment, the invention simplifies the removal of a needle shield, providing the possibility to remove it by the one-hand technique.

In another embodiment, the invention provides the needle shield design that may be similar for all kinds of devices having a sharp needle.

In another embodiment, the invention provides a universal method for the needle shield removal for all kinds of devices having a sharp needle.

In another embodiment, the invention prevents device damage during shield removal.

In another embodiment, the invention simplifies the design of the shield.

### BRIEF DESCRIPTION OF THE DRAWINGS

FIGS. 1 to 10 show the first embodiment of the present invention where the needle subassembly is a catheter introducer and needle shield made as a single detail.

FIG. 1 shows a general top view of the device.

FIG. 2 shows a general side view of the device.

FIG. 3 is a cross sectional view taken substantially along lines 3-3 in FIG. 1.

FIG. 4 is a cross sectional view taken substantially along lines 4-4 in FIG. 3.

FIG. 5 is a cross sectional view taken substantially along lines 5-5 in FIG. 2.

FIGS. 6-10 show the stages of the needle shield removal.

FIGS. 11 to 14 show the second embodiment of the present invention.

FIGS. 11, 12 show top view and cross sectional views, respectively, of the needle shield before usage.

FIGS. 13, 14 show a cross sectional and a top views, respectively, of the needle shield after usage.

FIGS. 15 to 20 show the third embodiment of the present invention where needle subassembly is a syringe equipped with a hypodermic needle, and the needle shield includes two extruded details.

FIG. 15 shows a cross sectional view of the device with an actuator in extended position.

FIGS. 16, 17 and 18 are cross sectional views taken along lines 16-16, 117-17 and 18-18, respectively, in FIG. 15.

FIG. 19 shows a cross sectional view of the device with an actuator in retracted position.

FIG. 20 shows the stage of the needle shield removal when it is fully separated from the syringe.

FIGS. 21 to 27 show the fourth embodiment of the present invention where needle subassembly is a hypodermic needle and the needle shield has a springy actuated lock mechanism.

FIG. 21 shows a cross sectional view of the device before usage.

FIG. 22 is a cross sectional view taken along line 22-22.

FIG. 23 shows a partial cross sectional view of the device before usage.

FIG. 24 shows a cross sectional view of the syringe equipped with the device from FIG. 21.

FIG. 25 shows a cross sectional view of the syringe equipped with the device from FIG. 23.

FIG. 26 shows the stage of the needle shield removal when it is fully separated from the syringe.

FIG. 27 shows a side view of the protected needle after usage.

### DETAILED DESCRIPTION OF THE INVENTION

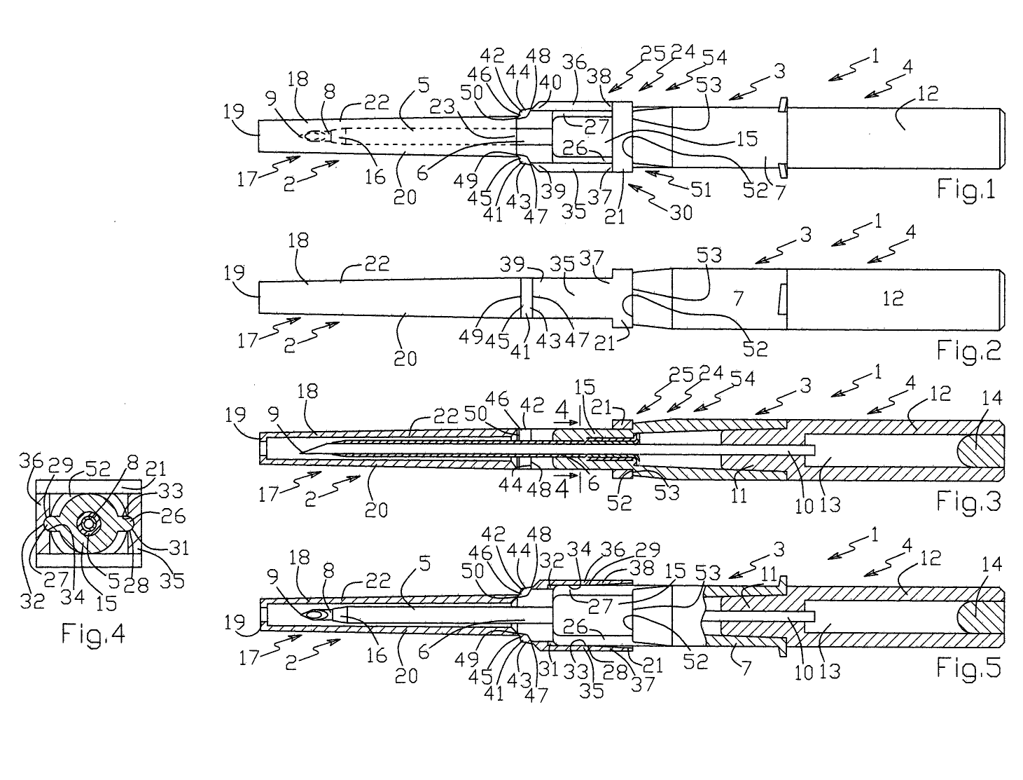

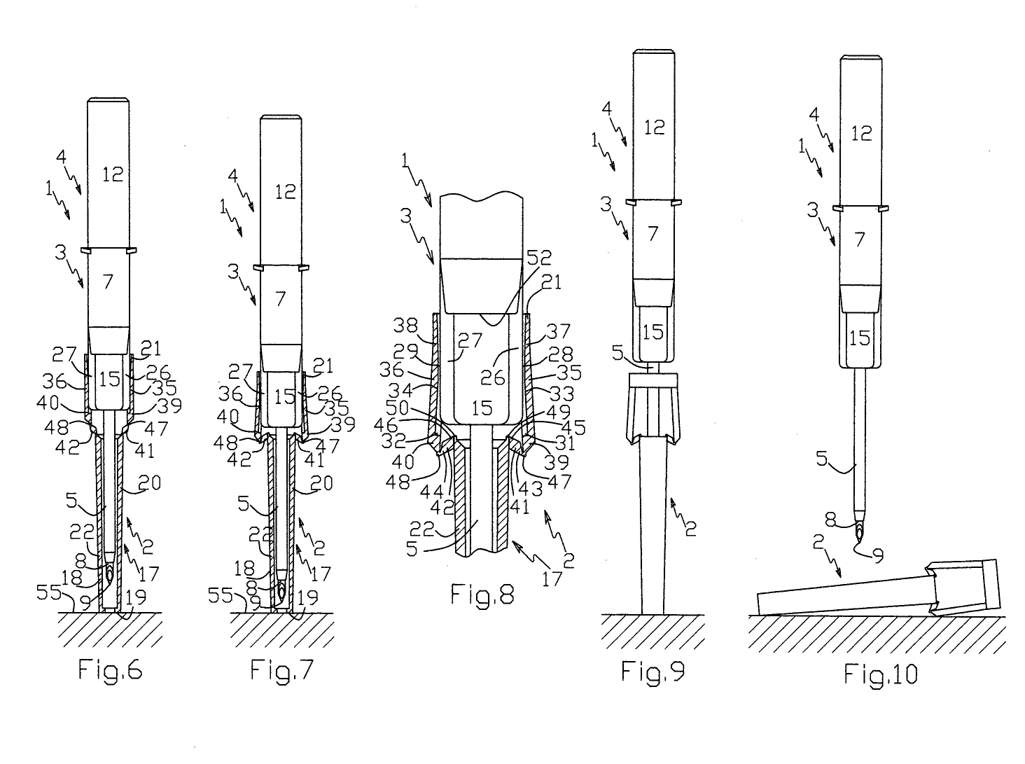

The first embodiment of a puncturing device with a needle shield constructed in accordance with the present invention is shown in FIGS. 1-10.

The device includes a needle shield 2 and a needle subassembly 1 in the form of a catheter introducer.

The catheter introducer 1 comprises a catheter 3 detachably mounted on a needle unit 4. The catheter 3 includes a catheter tube 5 whose proximal end 6 is connected to a catheter hub 7. The needle unit 4 includes a needle 8 with a sharp distal end 9 and a proximal end 10 secured to the distal portion 11 of a handle 12. The handle 12 serves at the same time as a needle hub having a flash chamber 13 and vent 14. The needle 8 passes through the catheter hub 7 and the tube 5 so that the needle distal end 9 protrudes distally beyond the distal end 16 of the catheter tube 5. There is a seat portion 15 of the needle subassembly 1, on which the needle shield 2 is detachably mounted for enclosing the needle 8 until the device usage. This seat portion 15 is a part of the catheter hub 7. The needle shield 2 has an actuating means 17 for the detachment of the needle shield from the needle subassembly. The actuating means 17 has an actuator 18 with an actuator distal end 19 which protrudes distally beyond the sharp distal needle end 9. The needle shield 2 also comprises a hollow trunk portion 20 in which the needle 8 are enclosed and a base portion 21 that form proximal end of the needle shield 2. The trunk portion 20 and the actuator 18 form a trunk-actuator portion 22, having a trunk-actuator proximal end 23 and a trunk-actuator distal end which is, at the same time, the actuator distal end 19. There is a coupling means 24 for the detachable mounting of the needle shield 2 on the seat portion 15 including a seat coupling means 25 and a shield coupling means 30. The seat coupling means 25 is formed by the elements of the seat portion 15 and represents two longitudinal protrusions 26, 27 having seat gripping surfaces 28, 29 respectively. The shield coupling means 30 is formed by the elements of the needle shield 2 and represents two longitudinal grooves 31, 32 having base gripping surfaces 33, 34 respectively. When the coupling means 25, 30 stay in mutually engaged position, the shield 2 is detachably mounted on the needle subassembly 1.

To provide this mutual engagement between the seat and shield coupling means, the shield 2 has two flexible arms 35, 36 having inner sides where the shield gripping surfaces 33, 34, respectively, are placed. The flexible arms 35, 36 provide springy contact and friction between the shield gripping surfaces 33, 34 and seat gripping surfaces 28, 29, respectively, with the force sufficient to preclude unwanted removal of the shield 2 and to ensure the needle shield 2 to be in an attached state.

Each of the arms 35, 36 has a proximal end 37, 38 affixed to the base portion 21 and an arm distal end 39, 40 directed distally to the trunk-actuator portion 22. There are spacer bars 41, 42 between the flexible arm 35, 36 and the trunk-actuator portion 22 correspondently. Each spacer bar 35, 36 has a spacer bar proximal end 43, 44 and a spacer bar distal end 45, 46 correspondently. There are proximal hinges 47, 48 connecting spacer bars proximal ends 43, 44 to the arms distal ends 39, 40 correspondently and distal hinges 49, 50 connecting the spacer bar distal ends 45, 46 to the trunk-actuator portion 22, forming a bistable structure with a first stable state shown in FIGS. 1-6. To preclude unwanted movement of the shield 2 in proximal direction relatively the needle subassembly 1 there are a supporting means 51, including at a seat supporting surface 52 on the seat portion 15 and a base supporting surface 53 on the base portion 21, wherein the supporting surfaces 52, 53 abut to each other. There is also an actuator stopping means 54 for precluding said actuator movement in proximal direction after said coupling means disengagement, wherein the support means 51 fulfills the role of the actuator stopping means. FIGS. 1-3, 5-7 show device with the actuator 18 staying in extended position in which the actuator distal end 19 stay in the most remote position relative from the needle distal end 9 and in the same time the needle shield 2 stays in the attached state. The actuator 18 is movable from an extended position to a retracted position FIGS. 7, 8 by the force applied to said actuator distal end, wherein being moved to the retracted position, the actuator 18 provides disengagement of the shield coupling means 30 from the seat coupling means 25 and, thus, the transition of the needle shield 2 from the attached state to the detached state, and the length of the detaching movement of the actuator 18 from the extended position to the retracted one is significantly shorter than the length of the needle 8. Being in the detached state, the needle shield 2 can be removed from the needle subassembly 1 by the gravity force.

A user receives the device shown in FIGS. 1-3, 5, 6 in a sterile package. The needle shield 2 is made of a flexible plastic. The user opens the package and withdraws the device. Holding the device by the handle 12, the user presses the actuator distal end 19 against any rigid surface 55 and continues to press until the resistance abruptly rises. During the movement of the needle subassembly 1 to the trunk-actuator portion 22, the spacer bars 41, 42 pivot about the distal hinges 49, 50 so that the spacer bar proximal ends 43, 44, together with the arm distal ends 39, 40, move away and laterally, thus flexing said arms 35, 36 and separating the shield gripping surfaces 33, 34 from the seat gripping surfaces 28, 29. The result of the disengagement of the shield coupling means 30 from the seat coupling means 25 is the transition of the needle shield 2 from the attached state to the detached state shown in FIGS. 7, 8. The user lifts the needle subassembly 1 (FIG. 9) above the surface 55 at the distance exceeding the length of said shield to complete the separation of the shield 2 from the needle subassembly 1 by the gravity force. FIG. 10 shows the needle subassembly 1 fully separated from the needle shield 2. The catheter placement should be executed as usual.

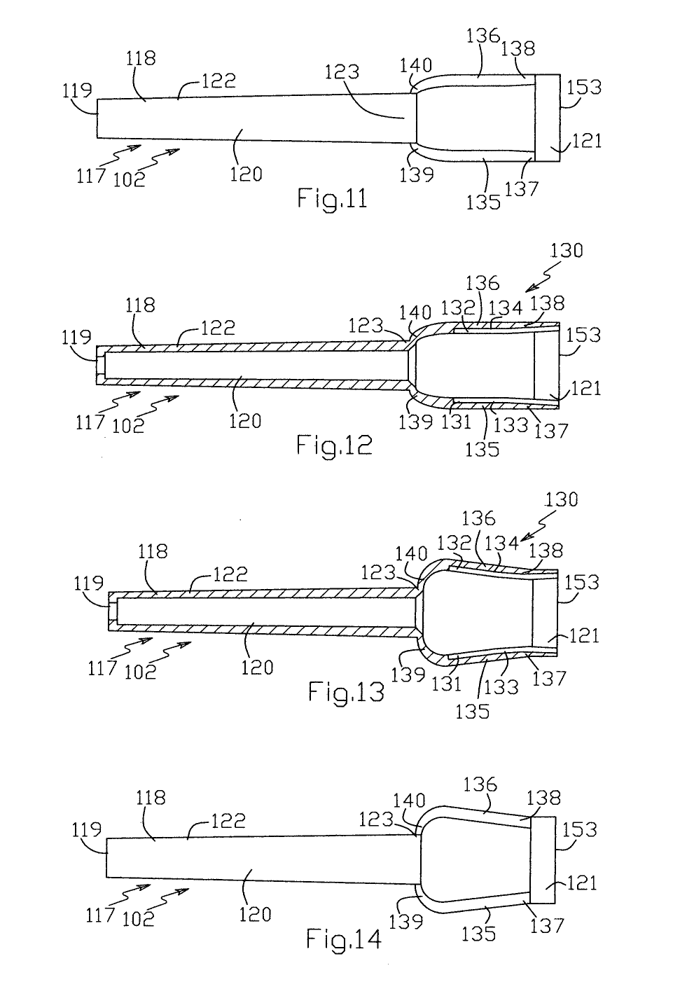

Another embodiment of needle shield 102 for a puncturing device in accordance with the present invention is shown in FIGS. 11-14.

The needle shield 102 is adapted to usage with needle subassembly 1, so further description relates only to differences between needle shield 1 and needle shield 102.

The needle shield 102 has an actuating means 117 for the detachment of said needle shield from said needle subassembly. The actuating means 117 has an actuator 118 having an actuator distal end 119. The needle shield 2 also comprises a hollow trunk portion 120 and a base portion 121 that form the proximal end of the needle shield 102. The trunk portion 20 and the actuator 18 form a trunk-actuator portion 122, having a trunk-actuator proximal end 123 and a trunk-actuator distal end, which is at the same time the actuator distal end 119. There is a shield coupling means 130. The shield coupling means 130 is formed by the elements of the needle shield 102 and presents two longitudinal grooves 131, 132 having base gripping surfaces 133, 134 respectively. The needle shield 102 has two flexible arms 135, 136 having inner sides where the shield gripping surfaces 133, 134 are placed, respectively.

Each of the arms 135, 136 has a proximal end 137, 138 affixed to the base portion 121 and an arm distal end 139, 140 directed distally to the trunk-actuator portion 122 and affixed to it.

To preclude unwanted movement of the shield 102 in proximal direction relatively to the needle subassembly, there is a base supporting surface 153. FIGS. 11, 12 show the shield in unstressed state, which corresponds to the state of the shield attached to needle subassembly. FIGS. 13, 14 show the shield in stressed state as a result of pressing the shield 102 against a rigid surface. This stressed state corresponds to the detached state of the shield 102 relatively to the needle subassembly. In such detached state, the shield 102 can be separated from the needle subassembly by the gravity force, as described above. The shield 102 is made of non-springy plastic in order to keep the stressed form (see FIGS. 13, 14) during its separation from the needle subassembly.

FIGS. 15 to 20 show the third embodiment of the present invention, where needle subassembly is a syringe equipped with a hypodermic needle and the needle shield including two extruded details.

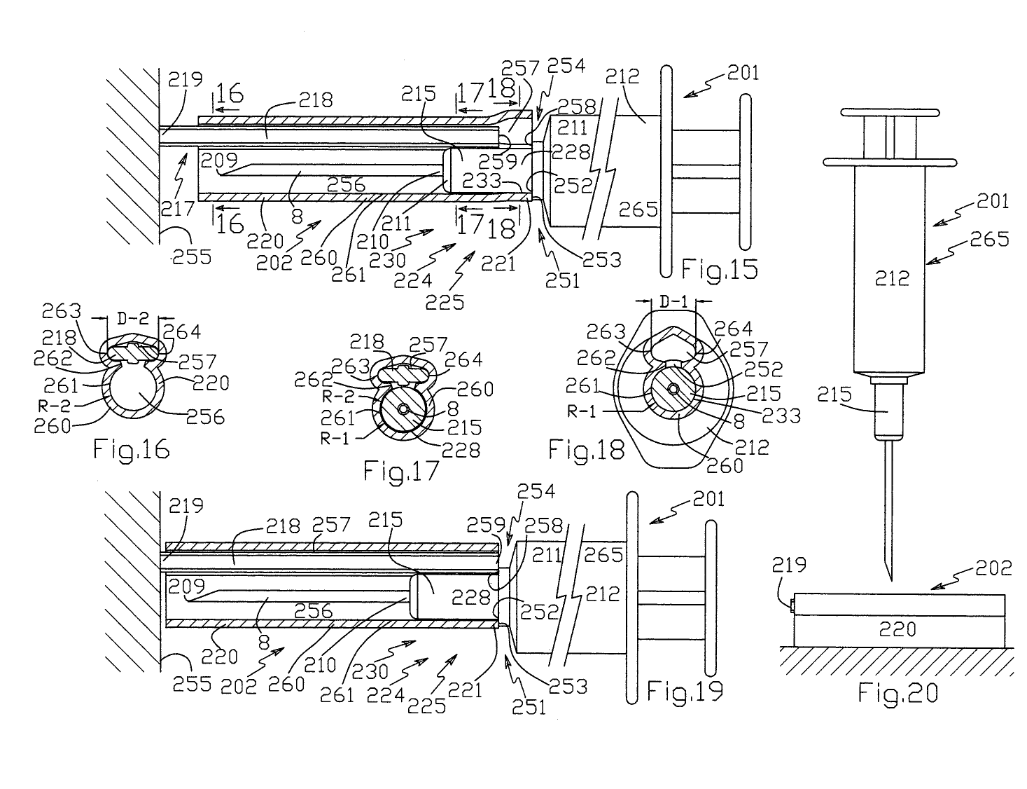

The device in FIG. 15 includes a needle subassembly 201 in the form of a syringe 265 with a hypodermic needle 208 and a needle shield 202. The syringe 265 serves as a handle 212. The needle 208 has a sharp distal end 209 and a proximal end 210 secured to the distal portion 211 of the syringe 265. There is a seat portion 215 of the needle subassembly 201 on which the needle shield 202 is detachably mounted for enclosing the needle 208 until the device usage. This seat portion 215 is a part of the syringe 265 and has mainly cylindrical form. The needle shield 2 has an actuating means 217 for the detachment of said needle shield from the needle subassembly 201. The actuating means 17 has an actuator 218 having an actuator distal end 219, which protrudes distally beyond said needle sharp distal end 209. The needle shield 202 also comprises a hollow trunk portion 220 in which the needle 208 is enclosed, and a base portion 221 that forms proximal end of the needle shield 202. There is a coupling means 224 for the detachable mounting of the needle shield 202 on the seat portion 215 including a seat coupling means 225 and a shield coupling means 230. The seat coupling means 225 represents a cylindrical seat gripping surfaces 228. The needle shield 202 includes the actuator 218 in the form of a bar and the trunk 220 having at least two longitudinal passageways: an actuator passageway 257 and a gripping passageway 256, whose inner surface 261 serves as a shield gripping surface 233. The curvature radius R-1 (FIG. 18) of the inner surface 261 of the gripping passageway in unstressed state provides a frictional engagement between the needle shield 202 and the seat portion 215. The passageways 256 and 257 are interconnected by a longitudinal slot 262 (FIGS. 16-18), forming a structure flexible in transversal directions. The inner transversal dimension D-1 (FIG. 18) measured between the points 263, 264 in the actuator passageway 257 on unstressed portion of the trunk 220 (shown on a cross sectional view taken along line 18-18) is shorter than the width (D-2) of the actuator bar 218 (FIGS. 16,17). So when the actuator bar 218 is moving along the actuator passageway 257, it enlarges the distance D-2 between points 263, 264 (see FIGS. 16, 17 which are the cross sectional views taken along line 16-16, 17-17 on the stressed part of the needle shield 202). The growth of the distance D-2 results in the growth of the curvature radius R-1 (FIG. 18) of the inner surface 261 of the gripping passageway 256 up to R-2 FIGS. 16 and 17, which is bigger than R-1 and thus results in separating the shield gripping surface 233 from the seat gripping surface 228 and consequently in detachment of the needle shield 202 from the needle subassembly 201. To preclude unwanted movement of the shield 202 in proximal direction relatively the needle subassembly 201 there are a supporting means 251, including at a seat supporting surface 252 on the seat portion 215 and a base supporting surface 253 on the base portion 221 FIG. 15, wherein the supporting surfaces 252, 253 abut to each other. There is also an actuator stopping means 254 for precluding said actuator movement in proximal direction after said coupling means disengagement. The actuator stopping means 254 include a proximal end 259 of the actuator 218 and the seat supporting surface 252.

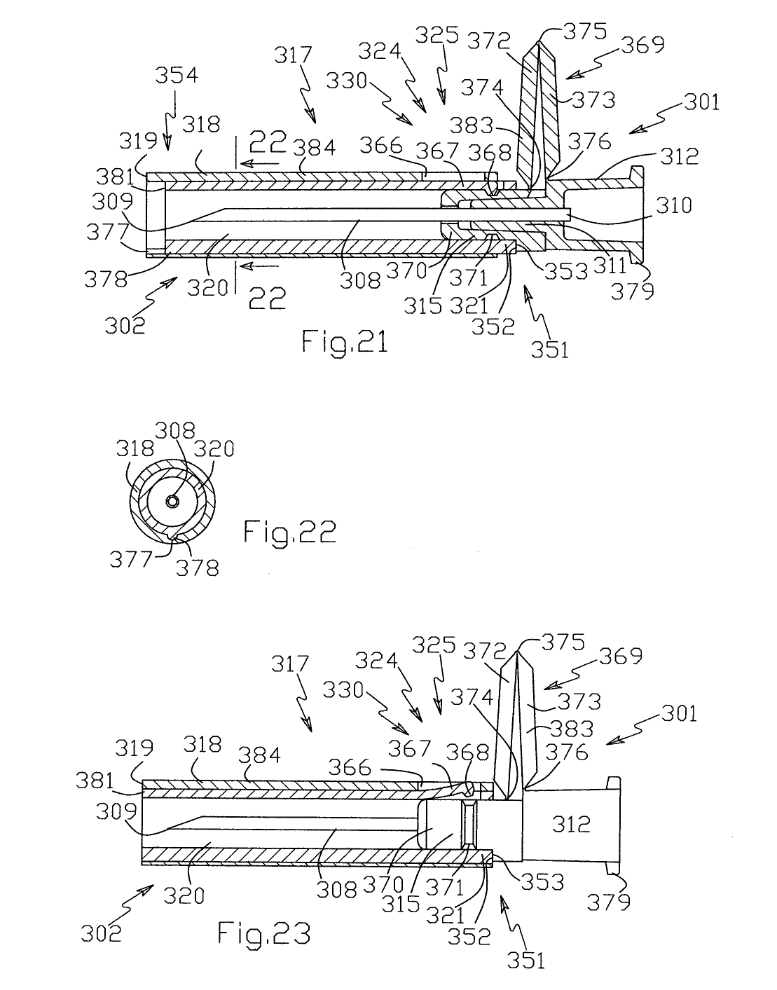

FIG. 15 shows the device with the actuator 218 staying in extended position in which the actuator distal end 219 stay in the most remote position relative from the needle distal end 209 and in the same time the needle shield 202 stay in the attached state. The actuator 218 is movable from the extended position to a retracted position, shown in FIG. 19, by the force applied to said actuator distal end, wherein being moved to the retracted position the actuator 218 disengages the shield gripping surface 233 from the seat gripping surface 228 FIG. 19 and thus the transition of the needle shield 202 from the attached state to the detached state, wherein the length of the detaching movement of the actuator 218 from the extended to the retracted position is significantly shorter than the length of the needle 208. And being in the detached state the needle shield 202 can be removed from the needle subassembly 201 by the gravity force FIG. 20.

The user removes the needle shield as described above.

FIGS. 21 to 27 show the fourth embodiment of the present invention, where needle subassembly is a hypodermic needle with protector and the coupling means has springy actuated lock mechanism.

The device according to FIGS. 21-27 includes a needle shield 302 and a needle subassembly 301.

The needle subassembly 301 includes a needle 308 and a protector 369. The needle 308 has a sharp distal end 309 and a proximal end 310 secured to the distal portion 311 of the handle 312, which is at the same time a needle hub. The protector 369 includes: a guard 370 connected with the handle 312 by a foldable link 383. The foldable link 383 consists of distal shoulder 372 and proximal shoulder 373 connected to each other by the middle hinge 375 and to guard 370 by the distal hinge 374 and to handle 312 by the proximal hinge 376. The guard 370 serves as a seat portion 315 on which the needle shield 302 is detachably mounted for enclosing the needle 308 until the device usage. The needle shield 302 comprises a hollow trunk 320, in which the needle 308 is enclosed, and a base portion 321 that forms proximal end of the needle shield 302. The needle shield 202 also has an actuating means 317 for the detachment of said needle shield from said needle subassembly. The actuating means 317 has a tubular actuator 318 placed outside the trunk 320 and coaxially to it. The actuator 318 further has an actuator distal end 319, which protrudes distally beyond said sharp distal needle end 309, and a trunk distal end 381. There is a coupling means 324 for the detachable mounting of the needle shield 302 on the seat portion 315 comprising: a seat coupling means 325 and a shield coupling means 330. The coupling means 324 made as a lock mechanism including the seat coupling means 325 represents a circular groove 371 on the guard 370 and the shield coupling means 330 in the form of a springy latch 367 with a protrusion 368 adapted to be engaged with the circular groove 371 when the springy latch 367 is loaded. To load the latch 367, it is pressed by the wall 384 of the actuator 318 (FIG. 21). There is a guide protrusion 378 (FIGS. 21,22) on the trunk 320 and a guide chase 377 corresponding to the former in the trunk wall 384 in order to preclude rotational movement of the actuator 318 relatively to the trunk 320. There is a window 366 in the wall 384.

To preclude unwanted movement of the shield 302 in proximal direction relatively to the needle subassembly 301, there is a supporting means 351 comprising a seat supporting surface 352 on the seat portion 315 and a base supporting surface 353 on the base portion 321, wherein the supporting surfaces 52, 53 abut upon each other. There is also an actuator stopping means 354 for precluding said actuator movement in proximal direction after said coupling means disengagement, and the trunk distal end 381 fulfills the role of the actuator stopping means 354.

FIGS. 21,24 show a device with the actuator 318 staying in extended position, in which the actuator distal end 319 stays in the most remote position relative to the needle distal end 309, and at the same time the needle shield 302 stays in the attached state. The actuator 318 is movable from the extended position to a retracted one, as shown in FIGS. 23,25, by the force applied to said actuator distal end, wherein being moved to the retracted position, the actuator 318 acts as a trigger releasing the latch 367 which flexes away from the guard 370 into the window 366. The latch release disengages the shield coupling means 330 from the seat coupling means 325 and brings the needle shield 302 from the attached state (FIGS. 21,24) to the detached state (FIGS. 23,25), wherein the distance of the detaching movement of the actuator 318 from the extended to the retracted position is significantly shorter than the length of the needle 308. And being in the detached state, the needle shield 302 can be removed from the needle subassembly 301 by the gravity force.

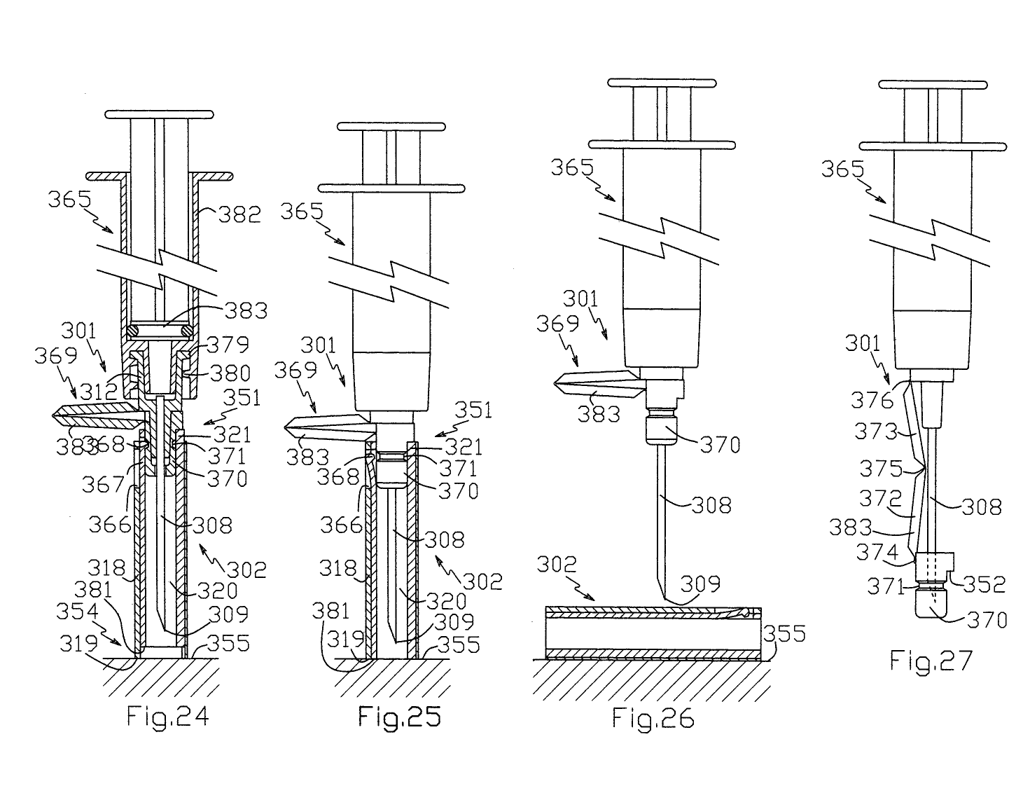

FIGS. 24-26 show the steps of the shield 302 removal.

In FIG. 24 the needle subassembly 301 with the shield 302 are shown attached by threads 379, 380 to a syringe 365. The syringe includes a plunger 383 and a barrel 382 which is used as a holder after the puncturing device 301 is attached. Holding the device by the barrel 382, the user presses the actuator distal end 319 against a rigid surface 355 and continues to press until the resistance abruptly rises. The movement of the needle subassembly results in the detachment of needle shield 302 from the guard 370. The user lifts the syringe 365 with the needle subassembly 301 above the surface 355 at the distance greater than the length of said shield to complete the separation of the shield 302 from the needle subassembly 301 by the gravity force. FIG. 26 shows the needle subassembly 301 fully separated from the needle shield 302. The syringe with the needle 308 is used as usual. The used needle 308 is protected by protector 369 (FIG. 27).

The principles, preferred embodiments and modes of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the Claims be embraced thereby.

1. Puncturing device with needle shield, comprising:
a needle subassembly having: a handle, and
a needle with a sharp distal end and a proximal end secured to said handle;
a needle shield detachably mounted on said needle subassembly for enclosing at least said needle distal end until the device usage, said needle shield having an actuating means for the detachment of said needle shield from said needle subassembly, said actuating means having at least one actuator with distal end which protrudes distally beyond said sharp distal needle end;
said needle shield having an attached state and a detached state, wherein being in said attached state said needle shield is attached to said needle subassembly with the force that precludes its unwanted detachment, and being in said detached state said needle shield can be removed from said needle subassembly by the gravity force,
said actuator has an extended position and a retracted position and is movable from said extended position to said retracted position by the force applied to said actuator distal end, wherein
in said extended position said actuator distal end stays in the most remote position relative to said needle distal end, and said needle shield stays in said attached state; and being moved to said retracted position, said actuator provides the transition of said needle shield from said attached state to said detached state, and the distance of the detaching movement of said actuator from said extended to said retracted position is significantly shorter than the length of said needle.

2. The device according to claim 1, includes
a handle distal portion to which said needle is secured;
a seat portion of said needle subassembly on which said needle shield is detachably mounted;
a coupling means for said detachable mounting of said needle shield on said seat portion including a seat coupling means that is formed by the elements of said seat portion, and a shield coupling means that is formed by the elements of said needle shield;
said coupling means staying in a mutually engaged position providing said detachable mounting of said needle shield on said needle subassembly, and said actuator being moved to said retracted position provides disengagement of said shield coupling means from said seat coupling means;
said needle shield including a hollow trunk portion in which at least said sharp distal end and the portion of said needle proximate to the latter are enclosed;
said trunk having a trunk distal portion that is proximate to said needle distal end and protrudes distally of it and a trunk proximal portion proximate to said shield coupling means.

3. The device according to claim 2, having: a supporting means and a base portion which is a part of said needle shield, wherein said supporting means including at least one seat supporting surface on said seat portion and at least one base supporting surface on said base portion, wherein said supporting surfaces abut upon each other precluding said base portion movement relatively to said seat portion in proximal direction during the actuator detaching movement.

4. The device according to claim 3, having an actuator stopping means for precluding said actuator movement in proximal direction after said coupling means disengagement.

5. The device according to claim 4, wherein said trunk portion and said actuator form a trunk-actuator portion.

6. The device according to claim 4, wherein said seat coupling means have at least one seat gripping surface and said shield coupling means have at least one shield gripping surface, and the engagement between said needle subassembly and said needle shield is provided by friction force between said seat and shield gripping surfaces, wherein said actuator being moved from said extended position to said retracted position causes said gripping surfaces to move away from each other.

7. The device according to claim 6, wherein
said trunk portion and said actuator form a trunk-actuator portion placed distally with respect to said base portion,
said base portion connected with said trunk-actuator portion by at least two flexible links, each of them having said gripping surface, a proximal link end connected to said base portion and a distal link end connected to said trunk-actuator portion, wherein the movement of said trunk-actuator portion from said extended position to said retracted position results in said links bending and therefore to the separation of said shield gripping surface from said seat gripping surface.

8. The device according to claim 6, having:
a trunk-actuator portion that is formed by said trunk portion and said actuator;
at least one flexible arm with said shield gripping surface, having an arm proximal end affixed to said base portion and an arm distal end directed distally to said trunk-actuator portion;
a spacer bar between said flexible arm and said trunk-actuator portion, having a spacer bar proximal end and a spacer bar distal end;
a proximal hinge connecting said spacer bar proximal end to said arm distal end and distal hinge connecting said spacer bar distal end to said trunk-actuator portion forming a bistable structure having the first stable state wherein during the movement of said trunk-actuator portion from said extended position to said retracted position said spacer bar pivots about said distal hinge, so that said spacer bar proximal end together with said arm distal end is moved away from said needle, flexing said arm and separating said shield gripping surface from said seat gripping surface, wherein said spacer bar being pivoted acts together with said supporting means as said actuator stopping means precluding further movement of said actuator.

9. The device according to claim 6, having said actuator in the form of a bar and said trunk having at least two longitudinal passageways, including a gripping passageway with inner wall forming said shield gripping surface and an actuator passageway; the passageways are interconnected by a longitudinal slot forming a structure flexible in transversal directions, so that said actuator bar being moved in said actuator passageway enlarges at least one transversal dimension of the gripping passageway, thus separating said shield gripping surface from said seat gripping surface.

10. A device according to claim 4, wherein said coupling means including a lock mechanism providing the engagement between said needle subassembly and said needle shield, and said actuator being moved from said extended position to said retracted position causes an unlocking in said lock mechanism.

11. The device according to claim 4, wherein said coupling means have at least one springy element which stays in loaded position until the movement of said actuator from said extended to said retraction position and is released as a result of said actuator movement, wherein said springy element provides disengagement of said shield coupling means from said seat coupling means, wherein said actuator works as a trigger.

12. The device according to claim 2 wherein said needle subassembly includes a catheter, said catheter having a catheter hub proximate to said distal portion of the handle, and a catheter tube with a distal end and a proximal end attached to said catheter hub; said needle passes through said catheter hub and tube, so that said needle distal end protrudes distally beyond said distal end of the catheter tube, wherein said catheter hub fulfils the role of said seat portion, so that said needle shield is detachably mounted on said catheter hub.

13. The device according to claim 2, including a needle hub to which said needle proximal end is secured, and said seat portion with said seat coupling means is a portion of said needle hub.

14. The device according to claim 2, including a syringe having a barrel with a distal portion, to which said needle is secured, and said barrel serves as said handle.

15. The device according to claim 2, including a protector means for enclosing at least said needle distal end after the device usage, wherein said needle shield is detachably mounted on the elements of said protector means.

16. A method of a removal of a needle shield from a puncturing device, wherein said device including a needle subassembly including a needle with a handle and a needle shield detachably mounted on said needle subassembly for enclosing said needle until the device usage, said method comprising the steps of:
holding said device in the area of said handle;
pressing said shield against a rigid surface;
orienting said device vertically so that said the shield is placed below said handle and above any surface at the distance greater than the length of said shield for the separation of said shield from said needle subassembly by the force of gravity.
